(19) United States
(12) Patent Application Publication
(10) Pub. No.: US 2022/0090185 A1
(43) Pub. Date: Mar. 24, 2022

(54) SYSTEM USING CONTAMINATION INDEX FOR EVALUATING FALSE POSITIVE DUE TO CONTAMINATION BY POSITIVE CONTROL TEMPLATE

(71) Applicant: ELECTRONICS AND TELECOMMUNICATIONS RESEARCH INSTITUTE, Daejeon (KR)

(72) Inventors: Eun-Soo JEONG, Daejeon (KR); Hye-Min LEE, Seoul (KR); Jae-Hyun AHN, Gwangju (KR)

(21) Appl. No.: 17/472,127

(22) Filed: Sep. 10, 2021

(30) Foreign Application Priority Data
Sep. 11, 2020 (KR) 10-2020-0117219

(51) Int. Cl. C12Q 1/6848; G01N 21/64

(52) U.S. Cl. CPC C12Q 1/6848; G01N 2021/6439; G01N 21/6428

(57) ABSTRACT

Disclosed herein is a method for determining a false positive by a real-time nucleic acid amplification reaction, including steps of a) preparing a positive control including a positive control gene including a target gene sequence and a contamination-determining gene sequence, b) obtaining a gene from a sample to prepare a group to be tested, followed by adding an internal control gene to the group to be tested, and c) adding probes capable of binding to each of a target gene, a contamination-determining gene and the internal control gene respectively to the positive control and the group to be tested, followed by proceeding a real-time nucleic acid amplification reaction (PCR), and characterized in that fluorescent light is emitted at the same wavelength when the probes capable of binding to each of the contamination-determining gene and the internal control gene are hydrolyzed.

### CROSS REFERENCE TO RELATED APPLICATION

The present application claims priority to Korean Patent Application No. 10-2020-0117219, filed on Sep. 11, 2020, the entire contents of which is incorporated herein for all purposes by this reference.

### BACKGROUND OF THE DISCLOSURE

### 1. Field of the Disclosure

The present disclosure relates to a method for determining a false positive, which can determine whether cross-contamination has been caused by a positive control nucleic acid template during molecular diagnosis using a fluorescent probe, such as real-time polymerase chain reaction, wherein this method may be accomplished by inserting a separate exogenous contamination-determining gene sequence used to determine whether cross-contamination occurred or not, which is distinguished from a target gene sequence, into the positive control template, and adding a fluorescent probe bound to this sequence complementarily into a sample reaction vessel for detecting the target to determine whether the false positive has been caused by contamination of a sample or a reagent by the positive control template.

### 2. Description of the Related Art

Nucleic acid amplification technology, such as polymerase chain reaction, is a method that can specifically amplify only a specific sequence portion in the genome, and is widely applied in various industries including medicine, agriculture, livestock, fisheries, pharmacogenetic tests, such as a diagnosis of infectious diseases like COVID-19 and non-infectious diseases like genetic diseases, blood screening tests, and forensic tests. Particularly, a real-time polymerase chain reaction method has recently been more widely used because it provides real-time measurement of a fluorescence intensity of a fluorophore, called a reporter, which is bound to a probe, according to a degree of amplification of a nucleic acid.

Real-time Polymerase Chain Reaction (real-time PCR), which is widely used among real-time nucleic acid amplification reactions, is based on a fluorescent light emission of a probe having a fluorophore between a forward primer and a reverse primer that complementarily bind to a template DNA, during a nucleic acid amplification process. A TaqMan® probe, which is most widely used in real-time polymerase chain reaction, is an oligomer composed of 25 to 30 bases, and has a reporter, which is a fluorescent substance, bound to 5′ end, and a quencher, which is a fluorescence quenching substance, bound to 3′ end thereof. The TaqMan® probe specifically binds to a target DNA failing to emit fluorescence by the quencher in annealing step, and in next step, that is extension step, the probe complementarily bound to the target DNA is hydrolyzed by a DNA polymerase which has an exonuclease function, whereby the reporter, whose fluorescent emitting has been suppressed by the quencher, emits fluorescence.

Molecular diagnostic tests using nucleic acid amplification should include a separate positive control reaction tube and a separate negative control reaction tube in parallel with a reaction tube for clinical sample examination and should be designed to detect an internal control target in the reaction tube containing a clinical sample to be tested as well. A template used as a positive control is generally manufactured by artificially synthesizing a target gene sequence to be detected and inserting the target gene sequence into a vector. In addition, during gene testing, a nucleic acid amplification reaction proceeds while the positive control target is added to the positive control reaction tube at constant concentration instead of the sample to be tested.

In molecular diagnostic test laboratories, it is common to repeatedly use many clinical samples over a long period of time, and there is always a possibility of generating invisible microscopic droplets during opening and closing a vessel containing a positive control template nucleic acid, or pipetting by technicians while such a molecular diagnostic test is carried out repeatedly over a long period, and such droplets can diffuse into a sample to be tested or a molecular diagnostic reagent. In addition, a target gene sequence in a positive control that has been subjected to a molecular diagnostic test has a theoretically 10\hat{ }12-fold increase in the number of targets compared with the number of targets before a nucleic acid amplification reaction, and contamination of a negative sample or a reagent by a positive control template due to many factors, such as improper treatment after testing and mistakes by technicians, will result in a false positive result in which a sample that should be judged as negative is judged as positive. As an example, false positive results have been reported several times in a COVID-19 diagnostic tests since a declaration of a pandemic by WHO in 2020.

To reduce a risk of cross-contamination by target nucleic acid amplification products among various causes of cross-contamination, a false positive due to a cross-contamination by nucleic acid products generated during amplification previously carried out can prevented significantly using deoxy-uridine triphosphate (dUTP), which is added during the polymerase chain reaction, instead of deoxy-thymidine triphosphate (dTTP) and Uracil-N-glycosylase (UNG), and cross-contamination can be also prevented by using a DNA removing solution before and after nucleic acid amplification reactions or carrying out clinical sample and positive control adding procedure in a place physically separated from a reagent preparation space. However, even if all methods to reduce or prevent such a cross-contamination are used, a possibility of cross-contamination by a positive control cannot be completely excluded, so it is required to immediately determine whether a false positive caused by the positive control occurred or not, during real-time nucleic acid amplification reaction.

### SUMMARY OF THE DISCLOSURE

The Sequence Listing created on Nov. 15, 2021 with a file size of 4.00 KB, and filed herewith in ASCII text file format as the file entitled “Sequence Listing_110HY2434US.TXT,” is hereby incorporated by reference in its entirety.

Accordingly, the present disclosure has been made keeping in mind the above problems occurring in the related art, and an objective of the present disclosure is to provide a method for determining a false positive by constructing a gene sequence in a positive control template that can determine whether a test sample is contaminated by a nucleic acid template of a positive control in molecular diagnosis by various real-time nucleic acid amplification reactions (ex. real-time PCR) using a fluorescent probe, and using the gene sequence.

Another objective of the present disclosure is to provide a method for determining a false positive without using a separate fluorescence channel other than a fluorescence channel used in a probe used for detecting a target during a diagnostic test using a real-time nucleic acid amplification reaction using a fluorescent probe.

In order to accomplish the above objectives, the present disclosure provides a method for determining a false positive by a real-time nucleic acid amplification reaction, including steps of a) preparing a positive control including a positive control gene including a target gene sequence and a contamination-determining gene sequence, b) obtaining a gene from a sample to prepare a group to be tested, followed by adding an internal control gene to the group to be tested, and c) adding probes capable of binding to each of a target gene, a contamination-determining gene and the internal control gene respectively to the positive control and the group to be tested, followed by proceeding a real-time nucleic acid amplification reaction (PCR), and characterized in that fluorescent light is emitted at the same wavelength when the probes capable of binding to each of the contamination-determining gene and the internal control gene are hydrolyzed.

According to an embodiment, the method of the present disclosure may further include a step of d) confirming that the real-time nucleic acid amplification reaction has been proceeded normally when fluorescent light is emitted at wavelength of a fluorophore bound to a probe bound to the internal control gene resulting from the nucleic acid amplification reaction of step c).

In addition, the method of the present disclosure may further include a step of e) determining that the group to be tested is contaminated by the positive control when more fluorescence intensity is confirmed at a wavelength at which a probe capable of binding to the internal control gene of the step d) emits fluorescent light.

According to a preferred embodiment of the present disclosure, the contamination-determining gene sequence is a DNA or RNA sequence having a length of 15 to 40 bp.

In addition, the present disclosure provides a method for determining a false positive by a real-time nucleic acid amplification reaction, wherein the probes are a nucleic acid oligomer having a fluorophore and a quencher bound thereto.

The probes of the present disclosure may be at least one probe selected from a group consisting of a TaqMan probe which is hydrolyzed by a forward primer or a reverse primer for amplification of a target gene sequence to generate fluorescence, a TaqMan MGB probe, a cycling probe which is cleaved by RNase H while binding to a complementary sequence, followed by being hydrolyzed by a primer to generate fluorescence, a Molecular Beacon which generate fluorescence while binding to a complementary sequence, a Scorpion probe, and a probe which includes a nucleic acid oligomer using a principle of FRET, but not limited thereto.

According to another embodiment of the present disclosure, a fluorophore bound to a probe complementary to an internal control gene is the same as a fluorophore bound to a probe complementary to a contamination-determining gene sequence or may be composed of a fluorophore having the same wavelength band as a fluorescent light-emitting fluorophore has.

According to another embodiment of the present disclosure, in detecting an internal control template in a diagnostic test through RT-qPCR or qPCR reaction, the time point (Ct value) at which a fluorescence value of the internal control increases again is 5 or more to 35 or less, and when the number of PCR cycles is in the range of 1 to 10 subsequently, an amount of probe for detecting the internal control can be adjusted so that the fluorescence value (FI, Fluorescence Intensity, or RFU, Relative Fluorescence Units) becomes constant and is maintained.

According to an embodiment of a method for determining a false positive by a real-time nucleic acid amplification reaction of the present disclosure, whether contamination is caused by a positive control or not can be determined based on an event where a fluorescence value (FI, Fluorescence Intensity, or RFU, Relative Fluorescence Units) used for detecting an internal control is kept constant and then increases again.

In addition, a method for determining a false positive by real-time nucleic acid amplification reaction is provided, wherein a degree of contamination may be evaluated semi-quantitatively on the basis of a time point at which the fluorescence value of an internal control first increases (Ct value), and the degree of contamination by a positive control template may be quantitatively calculated by Equation 1 below:

$${CI}_{PCT} = \frac{{F}_{IC \_ end} - {F}_{IC \_ st}}{{F}_{Target}} \qquad [ Equation 1 ]$$

When a positive control nucleic acid template suggested in the present disclosure, and a fluorophore having the same wavelength band as a reporter fluorophore has, which is used for detecting an internal control target in order to determine contamination by the positive control nucleic acid template during a target detection in a nucleic acid extract of a sample are used, it is possible to determine whether a sample or a reagent contaminated by the positive control nucleic acid template shows whether a result is a false positive or not based on a final fluorescence value higher than a fluorescence amplification signal while a designed internal control target amplifies, so it is also possible to improve accuracy and reliability of a diagnostic test via a nucleic acid amplification reaction.

According to an embodiment of the present disclosure, since it is possible not to add a separate primer for determining a false positive, and a fluorophore having the same wavelength band as a reporter fluorophore of a probe, which is used for detecting an internal control to detect an positive control, is used as a reporter, so it is also possible not to use a separate fluorescence channel required for detecting the positive control template in the design and development of molecular diagnostic reagents using multiplex detection and it has an advantage of not being limited by the number of multiplex detection targets.

According to an embodiment of the present disclosure, it is possible to quantitatively evaluate an extent to which contamination by a positive control template affects results, by calculating a contamination index.

### BRIEF DESCRIPTION OF THE DRAWINGS

The above and other objectives, features, and other advantages of the present disclosure will be more clearly understood from the following detailed description taken in conjunction with the accompanying drawings, in which:

FIG. 1 is a schematic diagram of a probe configuration using the same reporter fluorophore as an internal control has without using a separate fluorescence channel in order to detect a target gene sequence from a nucleic acid extracted from a sample, according to an embodiment of the present disclosure;

FIG. 2 is a schematic diagram showing relative positions of binding sites in a vector where a probe for contamination check binds complementarily, which is used as a positive control to be detected during a real-time nucleic acid amplification reaction when a sample or a reagent is contaminated by the positive control, according to an embodiment of the present disclosure;

FIG. 3 is a schematic diagram showing relative positions of a probe for contamination check in a vector, according to another embodiment of the present disclosure;

FIG. 4 is a schematic diagram showing relative positions of a probe for contamination check in a vector, according to another embodiment of the present disclosure;

FIG. 5 is an amplification curve of a target nucleic acid in a positive sample, which is predicted during a nucleic acid amplification reaction performed by using a TaqMan® probe, when a primer and a probe for detecting a positive control template and a target are constructed according to an embodiment of the present disclosure and there is no contamination of a sample or a reagent by the positive control template;

FIG. 6 is an amplification curve of a target nucleic acid in a negative sample, which is predicted during a nucleic acid amplification reaction performed by using a TaqMan® probe, when a primer and a probe for detecting a positive control template and a target are constructed according to an embodiment of the present disclosure and there is no contamination of a sample or a reagent by the positive control template;

FIG. 7 is an amplification curve of a target nucleic acid in a negative sample, which is predicted during a nucleic acid amplification reaction performed by using a TaqMan® probe, when a primer and a probe for detecting a positive control template and a target are constructed according to an embodiment of the present disclosure and there is a contamination of a sample or a reagent by the positive control template;

FIG. 8 is a schematic diagram showing a design of a positive control template prepared according to the present disclosure;

FIG. 9 is a real-time amplification curve of a target, which is experimentally confirmed after preparing a positive control template, a primer and a probe, followed by artificially contaminating a negative sample with the positive control, according to embodiments of the present disclosure; and

FIG. 10A-10D are a real-time amplification curve of a target, which is experimentally confirmed after contamination with a positive control template at a certain concentration in a positive sample having different concentrations of target gRNA occurred, according to embodiments of the present disclosure.

### DESCRIPTION OF THE PREFERRED EMBODIMENTS

Hereinafter, preferred embodiments will be described in detail so that those who ordinarily skilled in the art can easily practice the present disclosure with reference to the accompanying drawings. However, in describing a preferred embodiment of the present disclosure in detail, when it is determined that a detailed description of a related well-known function or configuration may unnecessarily obscure the gist of the present disclosure, the detailed description thereof will be omitted. Unless defined otherwise, all technical and scientific terms used herein have the same meaning as commonly understood by one of ordinary skill in the art to which this disclosure belongs. In addition, the same reference numerals are used throughout the drawings for parts having similar functions and actions.

The present disclosure will be described in detail as below.

In molecular diagnosis of diseases using real-time nucleic acid amplification reaction, a positive control (PC) reaction is performed in parallel to ensure accuracy and reliability of reagents used, and a Plasmid DNA in which target gene sequence to be confirmed is inserted is widely used as a positive control template.

As used herein, a “positive control” is a control in which a “target gene sequence” is present. According to the present disclosure, it is possible to analyze whether a group to be tested is positive or not by analyzing real-time polymerase chain reaction (PCR) results of the positive control and real-time polymerase chain reaction (PCR) results of the group to be tested. In addition, a “positive control gene sequence” of the present disclosure means a gene sequence used in the positive control.

As used herein, a “target gene sequence” refers to a specific gene sequence to be detected, and a “contamination-determining gene sequence” refers to a gene sequence to be inserted into a gene sequence of a positive control as a certain gene sequence and has a sequence different from the target gene sequence. In a control gene sequence, the “contamination-determining gene sequence” may be located on the side of the “target gene sequence”, between “a plurality of target gene sequences”, or within one “target gene sequence”, but is not limited thereto, and it may be modified and used according to a level of ordinary skill in the art.

As used herein, a “positive” means that a target gene sequence is present, and a “false positive” means that a test result is determined to be positive even though a target gene sequence does not exist. In the present disclosure, when a target gene sequence is present, expression (or fluorescent light emission) occurs at a position of the target gene sequence while real-time polymerase chain reaction (rt-PCR) proceeds, and when expression occurs at the position of the target gene sequence, a test result can be judged as “positive”.

As used herein, a “contamination” means that a gene sequence of a “positive control” from the air, experimental tool, or the like is introduced into a “group to be tested”. In this case, even if a target gene sequence does not exist in the “group to be tested”, a result of real-time polymerase chain reaction (rt-PCR) may appear as “positive”, which is called “a false positive”.

In molecular diagnostic tests, an “Internal Control (IC)” is also called as an Internal Positive Control (IPC) and is used to demonstrate a role of a reaction mixture for ensuring accurate nucleic acid amplification of a target by being extracted and amplified together with the target in a reaction tube containing the target such as pathogens. An “Internal control gene” means a gene used as an internal control, and any gene sequence can be used as the internal control gene, but the internal control gene is different from a contamination-determining gene sequence and a target gene sequence.

As used herein, a “probe” refers to a segment (or a fragment) complementary to a specific base sequence of DNA or RNA added to confirm the presence or absence of a specific gene sequence. In the present disclosure, the probe may bind to a fluorophore and a quencher. According to a general experimental method, the probe binds to a specific gene sequence, followed by being separated from the gene sequence while synthesis of the gene sequence initiates, and then the fluorophore and the quencher are separated from the probe, thereby displaying fluorescence. The presence or absence of the corresponding gene sequence can be determined by analyzing an intensity of fluorescence.

According to the present disclosure, the “probe” may be composed of sequences complementary to a target gene sequence, a contamination-determining gene sequence, and an internal control gene sequence, respectively.

In addition, according to the present disclosure, the probe capable of binding to each of the contamination-determining gene sequence and the internal control gene sequence may bind to the same fluorophore or a fluorophore having the same wavelength band.

According to the present disclosure, when the contamination-determining gene sequence does not exist in a group to be tested, that is, when a result is not a false positive, only a fluorescence intensity of a fluorophore bound to a probe complementary to an internal control gene appears, whereby a constant fluorescence intensity will be observed.

However, when the contamination-determining gene sequence is present in a group to be tested, that is, when a result is a false positive, a fluorescence intensity of a fluorophore bound to the probe complementary to an internal control gene will appear, along with a fluorescence intensity of a fluorophore bound to a probe complementary to a contamination-determining gene sequence. In this case, since the wavelength band of each fluorophore may be the same when a result is not false positive, a fluorescence intensity stronger than that of a fluorophore bound to a probe complementary to an internal control gene will appear, and a fluorescence may be observed in an escalating manner.

In molecular diagnosis of infectious diseases, when a contamination by a positive control template occurs even in a small proportion during a nucleic acid amplification reaction, such contamination can lead to a false positive reaction that results in a positive result even though a sample to be tested is not infected with a pathogen.

Accordingly, the present disclosure provides a method for determining whether a false positive occurs or not by inserting a contamination-determining gene sequence, which is a specific gene sequence, into a positive control, and detecting presence or absence of the contamination-determining gene sequence in a group to be tested.

Furthermore, in the present disclosure, an internal control is used to check whether the gene testing of a group to be tested is in progress or not, and it is possible to conclude that a reaction proceeded normally when an internal control gene sequence remains after the gene testing by introducing an internal control into the group to be tested.

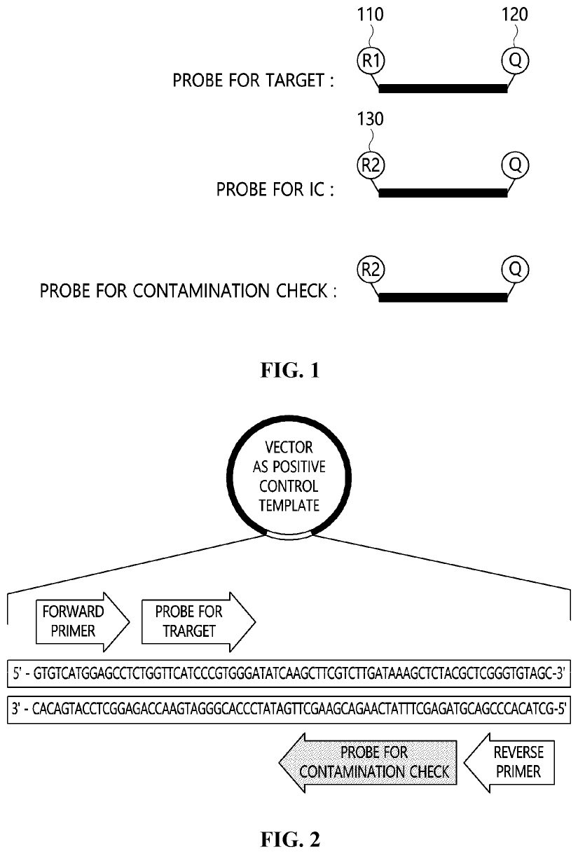

FIG. 1 is a view showing configurations of a probe for detecting a target gene during a real-time nucleic acid amplification reaction, a dual-labeled probe for detecting an internal control, and a dual-labeled probe for detecting contamination of a group to be tested, which is caused by a positive control, according to an embodiment of the present disclosure. 110 in FIG. 1 is a reporter for detecting a target, which is a fluorophore that emits fluorescence between 400 nm and 800 nm, and a reporter like FAM, HEX, TET, JOE, CY3, CY5, CAL Fluor560, CAL Flour610, ATT0565 NHS-ester, ROX NHS-ester, TexasRed NHS-ester, Yakima Yellow and the like is mainly used, but is not limited thereto. This reporter or fluorophore is covalently bonded to a 5′ end of a probe, a quencher is covalently bonded to the 3′ end of the probe, and when the quencher is present in close to the fluorophore in an excited state by a light source, fluorescence of the fluorophore is suppressed through Fluorescence Resonance Energy Transfer (FRET). Substances used as a quencher include, but are not limited to Black Hole Quencher (BHQ1, BHQ2, BHQ3), Blackberry Quencher (BBQ650), Dabcyl and Eclipse quencher, etc. Any substance that can suppress fluorescence from a fluorophore through FRET can be used.

In a probe for determining whether contamination by the positive control (PC) template occurs or not, a reporter having the same fluorescence wavelength band as a probe has, which is used for detecting an internal control, is used, but a separate primer is not used.

FIG. 2 shows a schematic diagram of a relative position of a target gene sequence to be included in producing a positive control template in a form of a plasmid using a vector during constructing a probe for detecting a target shown in FIG. 1, and a gene sequence to be complementarily bound to a probe for checking contamination. When a forward primer extends by an action of a Taq DNA polymerase, the physical distance between the forward primer and a quencher increases while a probe for detecting a target is hydrolyzed by an activity of a 5′ exonuclease, and a R1 reporter emits a fluorescence signal, and then a signal increases as the number of a nucleic acid amplification cycle increases. A probe used for confirming a contamination is preferably positioned in a direction in which a reverse primer extends, to make the probe be degraded during extension of the reverse primer.

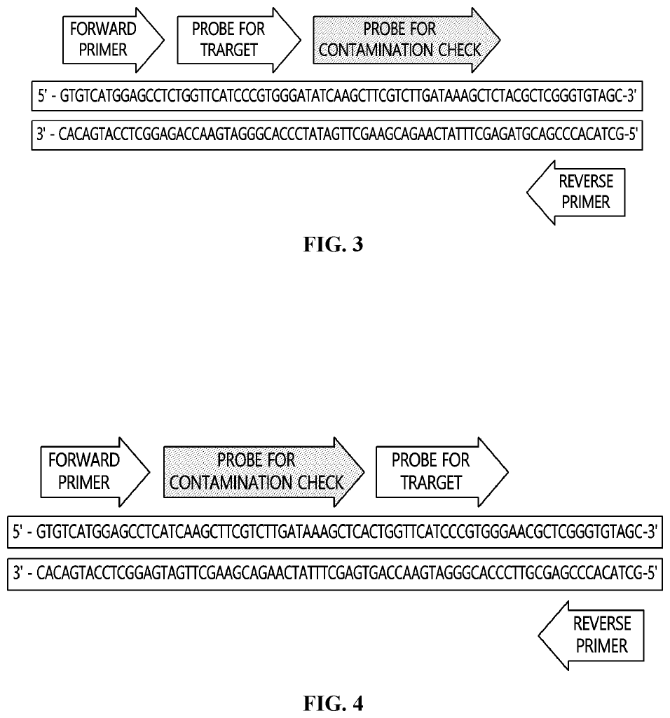

A relative position of a gene sequence to bind complementarily with a probe for confirming contamination, which is suggested herein, is not limited to a relative position shown in FIG. 2 and may be configured as shown in FIGS. 3 and 4. According to embodiments shown in FIGS. 2, 3 and 4, a contamination-determining gene sequence is characterized by being located between a forward primer and a reverse primer for detecting a target.

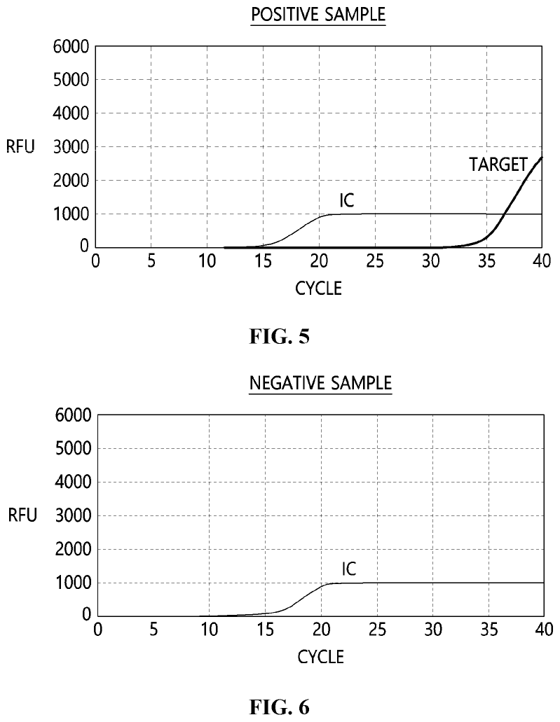

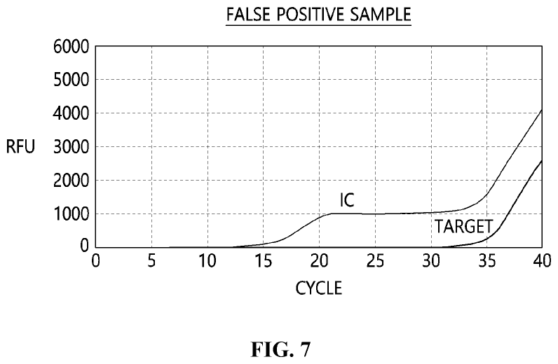

When a positive control template is prepared, and a primer and a probe for detecting a target are configured in a manner suggested herein, a fluorescence amplification curve of an internal control depicted in FIGS. 5 and 6 is shown in case that a sample or a reagent is not contaminated by the positive control template. However, when a sample or a reagent is contaminated by a positive control template, another increase in a nucleic acid amplification curve of an internal control, in which a constant fluorescence value appeared as depicted in FIG. 7, is demonstrated, and it is possible to determine whether or not a result is a false positive, depending on a shape of a nucleic acid amplification curve of an internal control even through a nucleic acid amplification curve appears while a target is detected.

More preferably, it is possible to quantitatively evaluate an extent to which contamination by a positive control template affects results using a contamination index (CI_{PCT}) described in Equation 1 designed by the present inventors:

$${CI}_{PCT} = \frac{{F}_{IC \_ end} - {F}_{IC \_ st}}{{F}_{Target}} \qquad [ Equation 1 ]$$

Wherein CI_{PCT} is a Contamination Index of Positive Control Template, F_{IC_end} is a fluorescence value in a final reaction cycle (RFU), F_{IC_st} is a fluorescence value of an internal control that initially reached a certain level (RFU), and F_{Target} is a final fluorescence value (RFU) of a target.

### Example 1. Construction of Primer Probe for Detecting SARS-CoV-2 for COVID-19 Diagnosis, Preparation of Positive Control Template, and Experiment for Simulating Contamination of Negative Sample by Positive Control Template

For detecting all subspecies of SARS-CoV-2, BLAST was carried out at NCBI for a S gene, and a region with a high detection rate due to a small number of mutations in NCBI registered sequence was selected, and then a primer and a probe was specified. A sequence of an internal control template (ICT) was designed as follows using an exogenous sequence that can avoid reaction with a primer and a probe for detecting SARS-CoV-2, and the primer and the probe for detecting SARS-CoV-2, and a probe sequence for confirming contamination are shown in Table 1 below.

ICT:
5′-ACCACTTAGCTTGAGCACGAAGACAGACTGTCGTCGTCCGTCAGACT
TACGTAGGAGCACCAGGAATCT-3′

TABLE 1
Sequence Information of Primer Probe
used in Example 1

| Primer/Probe | Sequence (5′→3′) |
|---|---|
| Forward Primer | GGCACAGGTGTTCTTACTGAGT |
| for Target | |
| Reverse Primer | GTCTGTGGATCACGGACAG |
| for Target | |
| Probe for Target | AGTAGTGTCAGCAATGTCTCTGCCAA |
| Forward Primer | ACCACTTAGCTTGAGCACGA |

for IC

| | |
|---|---|
| Reverse Primer | AGATTCCTGGTGCTCCTACG |

for IC

| | |
|---|---|
| Probe for IC | ACAGACTGTCGTCGTCCGTCAGACT |
| Probe for | ACATAACGCCCGGGATAACAGAGCTG |
| contamination check | |

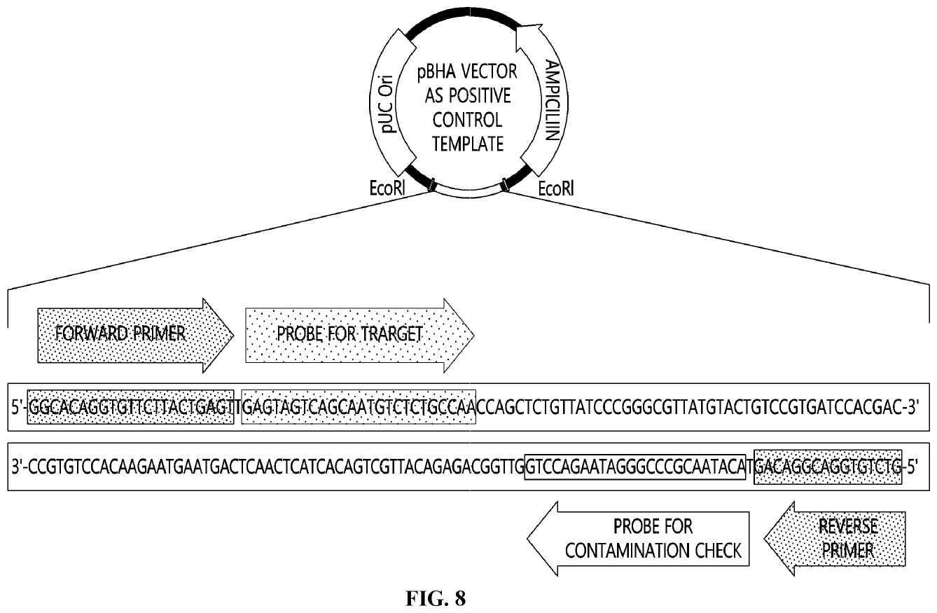

As shown in FIG. 8, a positive control template was inserted into the pBHA Vector by constructing a primer and a probe for target, and a probe sequence for confirming contamination.

A probe used in this Example is a TaqMan probe, and in this example, the TaqMan probe, which is synthesized by Neoprobe in South Korea through HPLC purification, was used.

TABLE 2
Construction of reporter and quencher of probe used in Example 1

| Probe | 5′ Reporter | 3′ Quencher |
|---|---|---|
| Probe for Target | FAM | BHQ1 |
| Probe for IC | Cal Red 610 | BHQ2 |
| Probe for Contamination Check | Cal Red 610 | BHQ3 |

In this Example, contamination of a reaction solution was simulated by intentionally adding 5 μl of a positive control template at a concentration of 10 copy/μl into the reaction solution during real-time RT-PCR using a negative sample, and a composition of the reaction solution is shown in Table 3.

TABLE 3
Composition table of reaction solution for simulating
contamination due to PCT according to Example 1

| | | |
|---|---|---|
| Component | Concentration | Volume (μl) |

| | | |
|---|---|---|
| 2x Master Mix for RT-qPCR | — | 10 |

| | | | |
|---|---|---|---|
| Forward Primer for Target | 20 | μM | 0.5 |
| Reverse Primer for Target | 20 | μM | 0.5 |
| Probe for Target | 5 | μM | 0.5 |
| Forward Primer for IC | 3 | μM | 0.5 |
| Reverse Primer for IC | 3 | μM | 0.5 |
| Probe for IC | 1 | μM | 0.5 |
| Probe for Contamination Check | 5 | μM | 0.5 |
| ICT (Internal Control Template) | 10⁷ | copy/μl | 1 |
| PCT (Positive Control Template) | 10 | copy/μl | 5 |

| | | |
|---|---|---|
| D.W. | — | 0.5 |
| Total | | 20 |

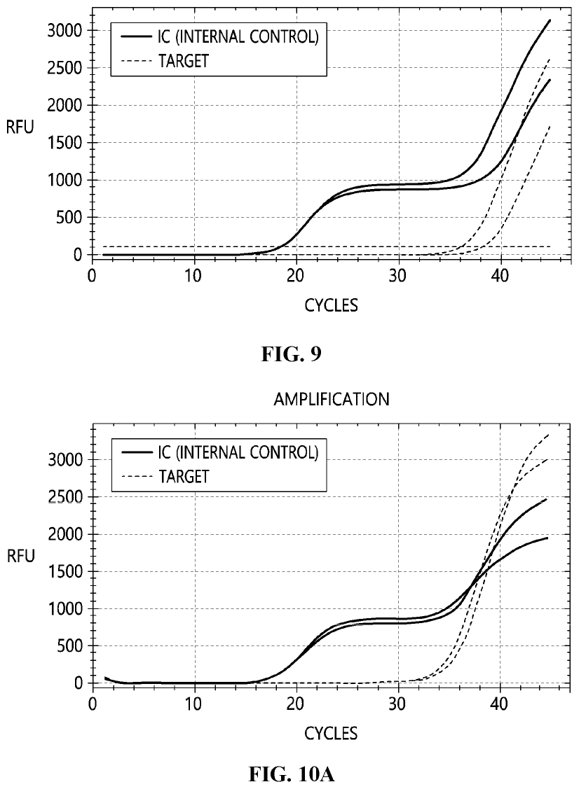

RT-qPCR was performed according to the reaction process provided in Table 4 using Bio-Rad CFX96™ Touch equipment, and results are shown in FIG. 9.

TABLE 4
RT-qPCR reaction process according to Example 1

| Step | Temperature | Time | | Number of cycles |
|---|---|---|---|---|
| Reverse | 50° C. | 20 | min | 1 |
| Transcription | | | | |
| Initial Denaturation | 95° C. | 10 | min | 1 |
| Denaturation | 95° C. | 15 | sec | 45 |
| Annealing and | 60° C. | 30 | sec | |
| Extension | | | | |

As predicted in FIG. 7, when the reaction solution was contaminated by the positive control template, a nucleic acid amplification curve of the internal control showing a constant RFU escalated again, and a phenomenon showing a higher RFU value might have been observed (refer to FIG. 9), and it was possible to confirm contamination by the positive control template sufficiently without using additional fluorescence for confirming contamination.

### Example 2. Simulating Contamination of Positive Sample by Positive Control Template

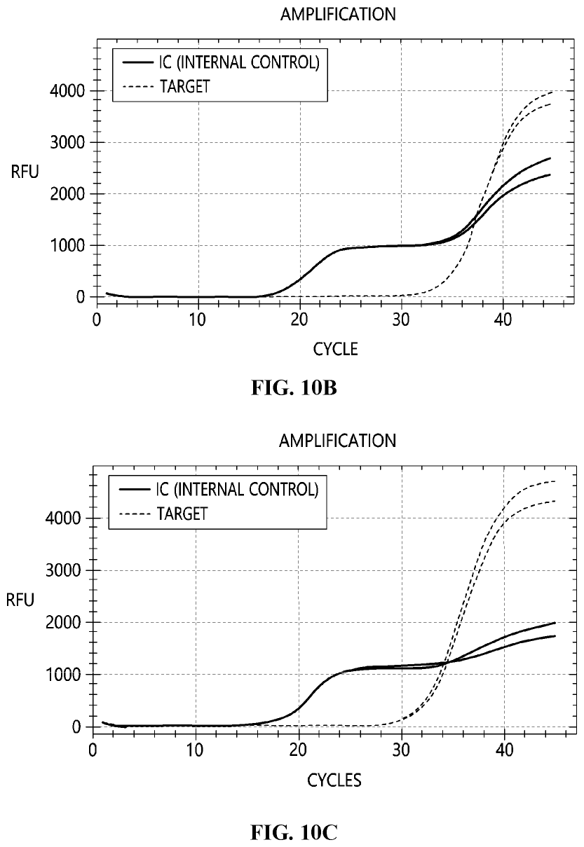

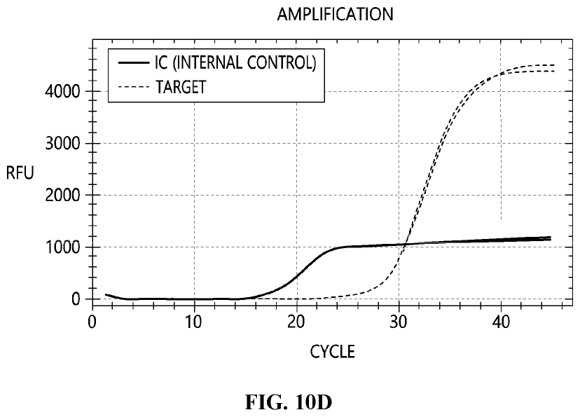

During diagnosis using real-time polymerase chain reaction was proceeding in a laboratory, a positive sample as well as a negative sample may be contaminated by a positive control template. A target amplification curve of the positive sample when contamination by the positive control template occurred is shown in FIG. 10A-10D, wherein AMPLIRUN® CORONAVIRUS SARS-CoV-2 RNA purchased from VIRCELL was used for simulating contamination of the positive sample. Other experimental conditions were the same as in Example 1, and concentrations of SARS-CoV-2 RNA and the positive control template contained in the sample are shown in Table 5.

TABLE 5
Concentration of SARS-CoV-2 RNA and positive control
template in reaction solution for simulating contamination
of positive sample by positive control template

| # | Positive Control Template | SARS-CoV-2 RNA | |
|---|---|---|---|
| a | 5 copy/μl | 5 × 10 | copy/μl |
| b | 5 copy/μl | 5 × 10² | copy/μl |
| c | 5 copy/μl | 5 × 10³ | copy/μl |
| d | 5 copy/μl | 5 × 10⁴ | copy/μl |

Judging from reaction results of a-d shown in FIG. 10A-10D, when a positivity (a degree of infection in the case of COVDI-19) of a sample became higher, there was a trend that a degree of increase in RFU value of the internal control showing a certain level due to contamination by the positive control template rises slightly again, and it would be reasonable to judge a test result as true positive even if there is a little contamination by the positive control template judging from the fact that there is another increase in RFU value of the internal control.

### Example 3. Quantitative Assessment of Contamination Using Contamination Index

Based on results of Examples 1 and 2, a contamination index (CI_{PCT}) was calculated according to formula for determining a degree of contamination by a positive control template using a RFU value of an internal control and a RFU value of a target for detection, and was shown in Table 6.

TABLE 6
CI_{PCT} according to an amount of target gene and relative
amount of contaminated positive control template

| Example # | Target conc./PCT conc. | CI_{PCT} |
|---|---|---|
| 1 | 0 | 0.893019 |
| 2(a) | 10 | 0.423811 |
| 2(b) | 100 | 0.396939 |
| 2(c) | 1,000 | 0.177335 |
| 2(d) | 10,000 | 0.04226 |

Judging from results of Table 6, when a negative sample was contaminated by the positive control template, the CI_{PCT} value was calculated to be 0.893. This is thought to be influenced by a hybridization efficiency of a primer and a probe for a target and a relative fluorescence efficiency of a reporter covalently bound to the probe, and when optimization is performed by adjusting concentrations of the primer and the probe, it is thought that the CI_{PCT} value would converge to a theoretical value of 1.

The largest value of CI_{PCT} in Example 2, which simulates the situation in which a positive sample was contaminated by a positive control sample, was 0.42, and we could recognize that the effect of a positive control template that contaminated the positive sample became meaningless as CI_{PCT} value converges to 0. When a contamination index obtained from a contamination caused by the positive control template was used, it would be possible to present criteria for determining a false positive (for example, in the case that CI_{PCT} value is 0.5 or higher, we can strongly suspect a false positive, so test should be carried out again), and this can increase the accuracy and reliability of molecular diagnostic kits.

Disclosed herein is a method for effectively evaluating a false positive reaction that occurs when a sample or a reagent is contaminated by a positive control (PC) template during molecular diagnosis using a real-time nucleic acid amplification reaction. The method is characterized in that a gene sequence for checking contamination is inserted in a gene sequence used to detect a target included in the positive control template. When constructing a primer and a probe used in molecular diagnosis using samples, a fluorescent substance (fluorophore) that is the same as or similar to a fluorescent substance emitting fluorescent light in a wavelength band used to detect the internal control, instead of using an additional primer and fluorescence channel for determining contamination by the positive control template. Therefore, it is possible to use all fluorescence channels available in molecular diagnostic equipment for target detection when multiple targets react simultaneously in the same reaction tube. In addition, it is possible to evaluate the degree of contamination more precisely quantitatively through calculation of a contamination index (CI_{PCT}) of the positive control template.

Although the present disclosure has been illustrated and described with reference to preferred embodiments as described above, it is not limited to the above-described embodiments, and various changes and modifications will be able to be made by those of ordinary skill in the art to which the present disclosure pertains within the scope not departing from the purpose of the present disclosure.

What is claimed is:

1. A method for determining a false positive by real-time nucleic acid amplification reaction using a fluorescent probe, the method comprising:
a) preparing a positive control including a positive control gene including a target gene sequence and a contamination-determining gene sequence,
b) obtaining a gene from a sample to prepare a group to be tested, followed by adding an internal control gene to the group to be tested, and
c) adding probes capable of binding to each of a target gene, a contamination-determining gene, and the internal control gene, to the positive control and the group to be tested, followed by proceeding the real-time nucleic acid amplification reaction (PCR),
wherein fluorescent light is emitted at the same wavelength when the probes capable of binding to each of the contamination-determining gene and the internal control gene are hydrolyzed.

2. The method of claim 1, further comprising d) confirming that the real-time nucleic acid amplification reaction has been proceeded normally when fluorescent light having is emitted at wavelength of a fluorophore bound to a probe bound to the internal control gene resulting from the nucleic acid amplification reaction of step c).

3. The method of claim 2, further comprising e) determining that the group to be tested is contaminated by the positive control when more fluorescence intensity is confirmed at a wavelength at which a probe capable of binding to the internal control gene of the step d) emits fluorescent light.

4. The method of claim 1, wherein the contamination-determining gene sequence is a DNA or RNA sequence having a length of 15 to 40 bp.

5. The method of claim 1, wherein the probes are a nucleic acid oligomer having a fluorophore and a quencher bound thereto.

6. The method of claim 5, wherein the probes are at least one probe selected from the group consisting of a TaqMan probe which is hydrolyzed by a forward primer or a reverse primer for amplification of a target gene sequence to generate fluorescence, a TaqMan MGB probe, a cycling probe which is cleaved by RNase H while binding to a complementary sequence, followed by being hydrolyzed by a primer to generate fluorescence, a Molecular Beacon which generate fluorescence while binding to a complementary sequence, a Scorpion probe, and a probe which includes a nucleic acid oligomer using a principle of FRET.

7. The method of claim 1, wherein a fluorophore bound to a probe complementary to the internal control gene is the same as a fluorophore bound to a probe complementary to the contamination-determining gene sequence or is composed of a fluorophore having the same wavelength band as the wavelength band of fluorescent light-emitting fluorophore.

8. The method of claim 6, wherein in detecting a internal control template in a diagnostic test through RT-qPCR or qPCR reaction, a time point (Ct value) at which a fluorescence value of the internal control increases again has a value in a range of 5 to 35, and thereafter, before or when the number of PCR cycles reaches 10, an amount of probe for detecting the internal control is adjusted so that the fluorescence value (FI, Fluorescence Intensity, or RFU, Relative Fluorescence Units) reaches a predetermined level and remains constant at the level.

9. The method of claim 8, wherein whether contamination is caused by the positive control or not is determined by an event where the fluorescence value (FI, Fluorescence Intensity, or RFU, Relative Fluorescence Units) used for detecting the internal control is kept constant and then increases again.

10. The method of claim 9, wherein a degree of contamination is evaluated semi-quantitatively on the basis of a time point at which the fluorescence value of the internal control first increases (Ct value) again, and
an extent to which contamination by a positive control template affects results is quantitatively calculated by Equation 1 below:
$${CI}_{PCT} = \frac{{F}_{IC \_ end} - {F}_{IC \_ st}}{{F}_{Target}} \qquad Equation 1$$


SEQUENCE LISTING (12 sequences)

SEQ ID NO: 1 - DNA, 69 bp, Artificial Sequence
  ICT
accacttagcttgagcacgaagacagactgtcgtcgtccgtcagacttacgtaggagcaccaggaatct

SEQ ID NO: 2 - DNA, 22 bp, Artificial Sequence
  Forward Primer for Target
ggcacaggtgttcttactgagt

SEQ ID NO: 3 - DNA, 19 bp, Artificial Sequence
  Reverse Primer for Target
gtctgtggatcacggacag

SEQ ID NO: 4 - DNA, 26 bp, Artificial Sequence
  Probe for Target
agtagtgtcagcaatgtctctgccaa

SEQ ID NO: 5 - DNA, 20 bp, Artificial Sequence
  Forward Primer for IC
accacttagcttgagcacga

SEQ ID NO: 6 - DNA, 20 bp, Artificial Sequence
  Reverse Primer for IC
agattcctggtgctcctacg

SEQ ID NO: 7 - DNA, 25 bp, Artificial Sequence
  Probe for IC
acagactgtcgtcgtccgtcagact

SEQ ID NO: 8 - DNA, 26 bp, Artificial Sequence
  Probe for Contamination Check
acataacgcccgggataacagagctg

SEQ ID NO: 9 - DNA, 74 bp, Artificial Sequence
  relative positions of binding sites for positive control
gtgtcatggagcctctggttcatcccgtgggatatcaagcttcgtcttgataaagctctacgctcgggtgtagc

SEQ ID NO: 10 - DNA, 74 bp, Artificial Sequence
  relative positions of a probe for contamination check in a vector
cacagtacctcggagaccaagtagggcaccctatagttcgaagcagaactatttcgagatgcgagcccacatcg

SEQ ID NO: 11 - DNA, 97 bp, Artificial Sequence
  positive control template
ggcacaggtgttcttactgagttgagtagtgtcagcaatgtctctgccaaccagctctgttatcccgggcgttatgtactgtccgtgatccacagac

SEQ ID NO: 12 - DNA, 97 bp, Artificial Sequence
  positive control template
ccgtgtccacaagaatgactcaactcatcacagtcgttacagagacggttggtcgagacaatagggcccgcaatacatgacaggcactaggtgtctg